SEQUENCE LISTING (6 sequences)

SEQ ID NO: 1 - DNA, 28 bp, Artificial Sequence
  Forward R77C
gccccggtggctctgctacacccagcgc

SEQ ID NO: 2 - DNA, 28 bp, Artificial Sequence
  Reverse R77C
gcgctgggtgtagcagagccaccggggc

SEQ ID NO: 3 - DNA, 31 bp, Artificial Sequence
  Forward C283Y
ggtgtgagcaccacgtaccaggagcacaacc

SEQ ID NO: 4 - DNA, 31 bp, Artificial Sequence
  Reverse C283Y
ggttgtgctcctggtacgtggtgctcacacc

SEQ ID NO: 5 - DNA, 34 bp, Artificial Sequence
  Forward E263K
gctcacagagcctctacaaaatctgtgtgtgtcc

SEQ ID NO: 6 - DNA, 34 bp, Artificial Sequence
  Reverse E263K
ggacacacacagattttgtagaggctctgtgagc

(19) United States
(12) Patent Application Publication
(10) Pub. No.: US 2023/0338369 A1
(43) Pub. Date: Oct. 26, 2023

(54) COMBINATION OF A PROTEASOME INHIBITOR AND A HDAC INHIBITOR AND ITS USE FOR THE TREATMENT OF GENETIC DISEASES LINKED TO A PROTEIN CONFORMATIONAL DISORDER

(71) Applicants: Centre d'Etude des Cellules Souches (CECS), Corbeil Essonnes (FR); INSTITUT NATIONAL DE LA SANTE ET DE LA RECHERCHE MEDICALE, Paris (FR); Genethon, Evry (FR); UNIVERSITE D'EVRY-VAL-D'ESSONNE, Evry (FR)

(72) Inventors: Lucile HOCH, Montigny Le Bretonneux (FR); Xavier NISSAN, Charenton-Le-Pont (FR); Isabelle RICHARD, Corbeil Essonnes (FR)

(21) Appl. No.: 17/789,128

(22) PCT Filed: Dec. 26, 2020
(86) PCT No.: PCT/EP2020/087895

§ 371 Date: Jun. 24, 2022

(30) Foreign Application Priority Data
Dec. 26, 2019 (EP) 19306772.5

(51) Int. Cl. A61K 31/4965; A61K 38/06; A61K 31/5355; A61K 31/167; A61K 31/18; A61K 31/422; A61K 45/06

(52) U.S. Cl. CPC A61K 31/4965; A61K 38/06; A61K 31/5355; A61K 31/167; A61K 31/18; A61K 31/422; A61K 45/06

(57) ABSTRACT

The present invention relates to the combination of a proteasome inhibitor and a histone deacetylase (HDAC) inhibitor and its use for the treatment of a genetic disease linked to a conformational disorder of at least one protein, said disorder causing the proteasome degradation of the protein.

### TECHNICAL FIELD

The present invention provides new pharmacological tools for treating genetic diseases linked to a conformational disorder of at least one protein, said disorder causing the cellular degradation of the protein through the proteasome pathway.

### STATE OF THE ART

During their maturation, the proteins undergo a folding process necessary for the formation of their tertiary and quaternary structures. In many genetic diseases, the folding of the implicated protein can be impacted by the amino acid changes resulting from genetic mutations (usually missense mutations). This defective folding can lead to their degradation by the ubiquitin proteasome system, although the protein may have at least partially retained a functional competence. This is the case for a certain number of genetic diseases whose protein matured in the endoplasmic reticulum such as cystic fibrosis, some retinitis pigmentosa, familial hypercholesterolemia, diabetes insipidus, Parkinson's disease linked to the parkin gene, phenylketonuria, Niemann-Pick disease, al-antitrypsin deficiency, some lysosomal diseases such as Fabry, Gaucher or Pompe disease and some muscular dystrophies.

Limb Girdle Muscular Dystrophies (LGMD) are a heterogeneous group of muscular dystrophies sharing common clinical presentation of muscle weakness affecting the shoulder and pelvic girdles. Within this group, sarcoglycanopathies are a subgroup caused by mutations in genes encoding the transmembrane protein sarcoglycan (SG) complex, located in the sarcolemma of striated muscle. Because the SG complex plays a key role in the maintenance of sarcolemma integrity during muscle contraction, mutations in these genes lead to muscular dysfunction. Four SGs have been described, namely, α-, β-, γ-, δ-SG, which cause different subtypes of LGMD: LGMD2D, LGMD2E, LGMD2C, LGMD2F corresponding to LGMD-R3, LGMD-R4, LGMD-R5 and LGMD-R6 with the recent nomenclature, respectively. While the clinical features of these LGMDs are well described, the molecular mechanisms associated with sarcoglycanopathies remain poorly understood. Analyses of large cohorts of patients have revealed that hundreds of different missense or null mutations of these genes can lead to sarcoglycanopathies, with the most frequent type being the arginine-to-cysteine substitution at the 77th amino acid (R77C) in α-SG. This mutation as well as other SG missense mutations lead to the production of a misfolded but still functional protein, recognized by the endoplasmic reticulum quality control (ERQC) system and degraded by the endoplasmic-reticulum-associated protein degradation (ERAD) pathway through the ubiquitin-proteasome system (Bartoli, M. et al., Human molecular genetics 17, 1214-1221 (2008); Gastaldello, S. et al., The American journal of pathology 173, 170-181 (2008); Soheili, T. et al., Hum Mutat. 33(2):429-39 (2012)).

Although no treatment is currently available for sarcoglycanopathies, recent studies suggest that inhibition of the different steps in the endoplasmic reticulum (ER) degradation system could be effective in restoring mutated SG expression in the plasma membrane (Bartoli, M. et al. (2008), Gastaldello, S. et al. (2008), Soheili, T et al. (2012)).

Evidence in support of this strategy has been described by different groups, as revealed by the positive impact of kifunensine when targeting mannosidase I activity (Bartoli, M. et al. (2008)) or the use of a proteasome inhibitor such as MG132 (Gastaldello, S. et al. (2008)), of thiostrepton (Hoch, L. et al., Sci Rep. 9(1):6915 (2019)) or of an autophagy inhibitor such as 3-methyladenine (WO2012/164234). Overall, these studies provide strong evidence that targeting cellular quality control or degradation pathways are of interest to rescue missense mutations that cause e.g. LGMD-R3, R4, R5 and R6.

However, there is still a need to find improved therapeutical approaches for treating said kind of genetic diseases especially in terms of efficacy and safety.

Document WO 2006/102557 generally discloses the use of protein degradation inhibitor(s) for treating a protein degradation disorder, especially cellular proliferation disorder such as cancer or protein deposition disorder.

### SUMMARY OF THE INVENTION

The inventors have shown that by combining a proteasome inhibitor, e.g. bortezomib, and a HDAC (histone deacetylase) inhibitor such as givinostat, it is possible to treat genetic diseases linked to a conformational disorder of at least one protein, said disorder causing the cellular degradation of the protein. Of particular interest is the fact that the quantity of proteasome inhibitor having potential toxicity can be drastically reduced. Moreover, the present application reports the efficiency of such a combination in the presence of various proteasome inhibitors, of various HDAC inhibitors, of various mutated proteins and of various mutations.

### Definitions

The definitions below represent the meaning generally used in the context of the invention and should be taken into account unless another definition is explicitly stated.

In the frame of the invention, the articles “a” and “an” are used to refer to one or several (i.e., at least one) of the grammatical object of the article. By way of example, “an element” means at least one element, i.e. one or more than one elements.

The terms “around”, “about” or “approximately” as used therein when referring to a measurable value such as an amount, a temporal duration and the like should be understood as encompassing variations of ±20% or ±10%, preferably ±5%, more preferably ±1%, and still more preferably ±0.10% from the specified value.

Intervals/ranges: throughout this disclosure, various aspects of the invention can be presented in the form of a value interval (range format). It should be understood that the description of values in the form of an interval is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from its natural environment or state. For example, an isolated nucleic acid or peptide is a nucleic acid or peptide which has been extracted from the natural environment in which it is usually found whether this be in a plant or living animal for example. A nucleic acid or peptide for example which is naturally present in a living animal is not an isolated nucleic acid or peptide in the sense of the invention whereas the same nucleic acid or peptide partially or completely separated from other components present in its natural environment is itself “isolated” in the sense of the invention. An isolated nucleic acid or protein can exist in substantially purified form, or can exist in a non-native environment such as, for example, a host cell.

In the context of the present invention, the following abbreviations for the commonly occurring nucleic acid bases are used: “A” refers to adenosine, “C” refers to cytosine, “G” refers to guanosine, “T” refers to thymidine, and “U” refers to uridine.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is an animal, preferably a mammal, more preferably a human. It may also be a mouse, a rat, a pig, dog or non-human primate (NHP), such as the macaque monkey.

In the sense of the invention, a “disease” or “pathology” is a state of health of an animal in which its homeostasis is adversely affected and which, if the disease is not treated, continues to deteriorate. Conversely, in the sense of the invention, a “disorder” or “dysfunction” is a state of health in which the animal is able to maintain homeostasis but in which the state of health of the animal is less favourable than it would be in the absence of the disorder. Left untreated, a disorder does not necessarily result in deterioration in the state of health of the animal over time.

A disease or disorder is “alleviated” (“reduced”) or “ameliorated” (“improved”) if the severity of a symptom of the disease or disorder, the frequency with which such a symptom is experienced by the subject, or both of these, is reduced. This also includes the disappearance of progression of the disease, i.e. halting progression of the disease or disorder. A disease or disorder is “cured” (“recovered”) if the severity of a symptom of the disease or disorder, the frequency with which such a symptom is experienced by the patient, or both, is eliminated.

In the context of the invention, a “therapeutic” treatment is a treatment administered to a subject who displays the symptoms (signs) of pathology, with the purpose of reducing or removing these symptoms. As used herein, the “treatment of a disease or disorder” means reducing the frequency or severity of at least one sign or symptom of a disease or disorder experienced by the subject. A treatment is said to be prophylactic when it is administered to prevent the development, spread or worsening of a disease, particularly if the subject does not have or does not yet have the symptoms of the disease and/or for which the disease has not been diagnosed.

As used herein, “treating a disease or disorder” means reducing the frequency or severity of at least one sign or symptom of a disease or disorder experienced by a subject. Disease and disorder are used interchangeably herein in the context of treatment.

In the sense of the invention, an “effective quantity” or an “effective amount” of a compound is that amount of compound which is sufficient to provide a beneficial effect to the subject to which the compound is administered. The expression “therapeutically effective quantity” or “therapeutically effective amount” refers to a quantity which is sufficient or effective to prevent or treat (in other words delay or prevent the development, prevent the progression, inhibit, decrease or reverse) a disease or a disorder, including alleviating symptoms of this disease or disorder.

### DETAILED DESCRIPTION OF THE INVENTION

The present invention relates to the use of a proteasome inhibitor and a HDAC (histone deacetylase) inhibitor for treating a genetic disease linked to a conformational disorder of at least one protein, said disorder causing the cellular degradation of the protein, in particular, its proteasomal degradation.

The present invention thus relates to the combination of a proteasome inhibitor and a HDAC inhibitor for use in the treatment of genetic diseases linked to a conformational disorder of at least one protein, said disorder causing the cellular degradation of the protein, in particular its proteasomal degradation.

In other words, a proteasome inhibitor and a HDAC inhibitor are used to prepare a medicament intended for the treatment of genetic diseases linked to a conformational disorder of at least one protein causing its cellular degradation, in particular its proteasomal degradation.

The invention thus relates to a method of treating genetic diseases linked to a conformational disorder of at least one protein, said disorder causing the cellular degradation of the protein, in particular its proteasomal degradation, comprising administering to a subject in need thereof, at an efficient dose, a proteasome inhibitor and a HDAC inhibitor.

The first active ingredient of a composition according to the invention is a proteasome inhibitor.

In the frame of the invention, a proteasome inhibitor is defined as a compound having proteasome inhibition activity. Such an activity can be evaluated by different methods known to the skilled person, e.g.:
by using the 20S Proteasome Activity Assay (Millipore) according to the instruction of the manufacturer;
by evaluating the chymotrypsin-like activity as disclosed in the examples, or the trypsin-like or caspase-like activity of the proteasome.

Such a proteasome inhibitor is advantageously chosen in the following list: bortezomib (or Velcade or PS-341), MG115, MG132, MG262, MG110, lactacystin, epoxomicin, eponemycin, carfilzomib (Kyprolis), CEP-18770, MLN2238, ONX-0912, marizomib and omuralide. More advantageously, the proteasome inhibitor is bortezomib, MG132 or carfilzomib, especially bortezomib.

Alternatively or in addition, a cyclic thiopeptide, in particular thiostrepton, can be used as reported by Hoch, L. et al. (2019).

Said proteasome inhibitors are usually administered intraveneously or orally.

A second active ingredient of a composition according to the invention is an inhibitor of histone deacetylases or HDAC inhibitor (HDACi).

As known in the art, the inhibitors of histone deacetylases (HDAC inhibitors) are compounds able to modify the epigenome. Their level of action relates to covalent modifications of histones. Preferably, such modifications concern: the acetylation of lysine residues, the methylation of lysine residues and of arginine, the phosphorylation of threonine and serine residues, the ubiquitination and the sumoylation of lysine residues.

Preferably, such compounds are able to modulate the histone acetylation level, especially by acting on the histone modification enzymes either directly at the level of their activity, or genetically at the level of their expression. The histone acetylation level results from the activity of two antagonistic enzymes: histone deacetylases (HDAC), resulting in a repressed chromatin, and histone-acetyltransferases (HAT) which allow the gene expression.

More preferably, such compounds are able to inhibit the activity of the enzymes involved in the deacetylation of the histones.

There are several classes of HDAC inhibitors according to their inhibition mode and to the class of HDACs they target.

Such compounds may be of any nature, for example proteins, peptides, antibodies, chemical molecules, or nucleic acids (antisense oligonucleotides, siRNA, shRNA, ribozymes, . . . ).

Histone deacetylation inhibitors comprise:
hydroxamic acids or salts thereof:
   trichostatin A (TSA);
   belinostat (PXD101);
   Panobinostat (LBH589);
   Givinostat (ITF2357);
   Resminostat (4SC-201);
   Abexinostat (PCI-24781);
   Quisinostat;
   Ricolinostat (ACY-1215);
   Citarinostat (ACY-241);
   Practilinostat;
   CHR-3996;
   alpha compound 8;
   MC1568;
   Tubacin;
   Tubastatin;
   suberoylanilide hydroxamic acid (SAHA or vorinostat or MK063), having formula:

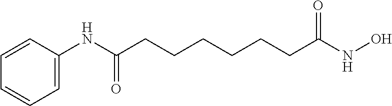 [SMILES: O=C(CCCCCCC(=O)Nc1ccccc1)NO]

cyclic tetrapeptides and depsipeptides:
   trapoxin B;
   apicidin;
benzamides:
   entinostat (MS-275), having formula:

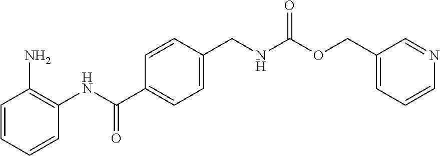 [SMILES: Nc1ccccc1NC(=O)c1ccc(CNC(=O)OCc2cccnc2)cc1]

CI-994;
106;
4SC-202;
Tacedinaline (C1994);
Mocetinostat (MGCD0103)
electrophilic ketones:
   trifluoromethyl ketones;
   α-cetoamides;
Aliphatic acid compounds:
   phenylbutyrate, having formula:

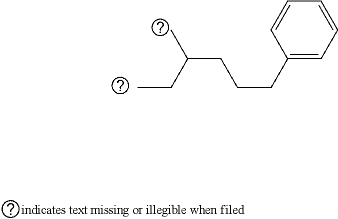

valproic acid or sodium valproate;
butyric acid
Dacinostat (LAQ824);
the other molecules:
nicotinamide;
cyclic tetrapeptides such as Romidepsin;
dihydrocoumarin;
naphthopyranone;
2-hydroxynaphaldehydes;
10-hydroxy-2-decenoic acid (10HDA);
SB939;
CUDC-101;
CUDC-907;
AR-42;
CHR-2845;
4SC-202;
CG200745;
Sulforaphane;
Kevetrin;
Apicidin;
Sodium butyrate;
(−)-Depudecin;
Sirtinol;
Cambinol;
Other Sirtuins inhibitors such as Ex-527;
N-Hydroxy-1,3-dioxo-1H-benz[de]isoquinoline-2(3H)-hexanamide or Scriptaid;
The hydroxamate derivative of butyric acid;
Isobutyramide;
CBHA (m-carboxycinnamic acid bishydoxyamide);
HC toxin;
M344 (4-dimethylamino-N-(6-hydroxycarbamoyl-hexyl)-benzamide);
Nullscript (4-(1,3-dioxo-1H,3H-benzo[de]isoquinolin-2-yl)-N-hydroxybutanamide);
PCI-34051, having formula:

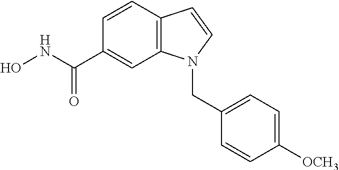 [SMILES: COc1ccc(Cn2ccc3ccc(C(=O)NO)cc32)cc1]

The chemical formulas of a number of these inhibitors are described in document Kazantsev and Thompson (Nature Reviews Drug Discovery, 7(10) 2008, 854-866).

Further, inhibitors of the methylation of histones may be:
SL11144, having formula:

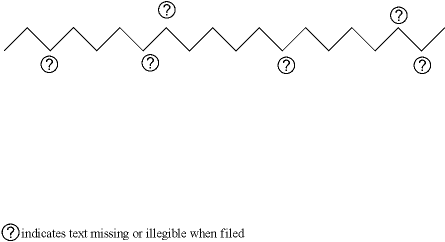 [SMILES: CCCCCCCCCCCCCCCCCCCC]

DZNep (3-Deazaneplanocin: inhibitor of S-adenosylhomocysteine hydrolase), having formula:

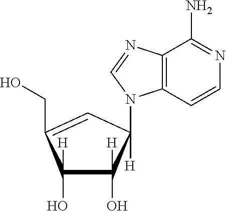

According to a preferred embodiment of the invention, the compound modifying the epigenome is a HDAC inhibitor.

As shown in the present application, the HDAC inhibitors are preferably those able to inhibit HDAC6. They can be pan-inhibitors, i.e. inhibitors of all types of HDACs. Alternatively, they can have a similar inhibitory action versus different HDACs including HDAC6, or a superior or even exclusive inhibitory action (selective inhibitor) versus HDAC6.

According to a specific embodiment, the HDAC inhibitor is givinostat (ITF2357), belinostat (PXD101) or panobinostat (LBH589), advantageously givinostat.

As an example of a HDAC6 inhibitor, tubacin can be used. Other examples are: ricolinostat (ACY-1215), tubastatin A or tubastatin A HCl, citarinostat (ACY-241), Nexturastat A, HPOB, SKLB-23bb, and WT161.

Further useful compounds are: TH34, scriptdroxinostat, BRD73954, CAY10603, ACY-738.

Said HDAC inhibitors are usually administered orally or possibly intravenously.

The proteasome inhibitor and the HDAC inhibitor may be administered simultaneously (e.g. in separate or unitary compositions) or sequentially in either order. In the latter case, the two compounds will be administered within a period and in an amount and manner that is sufficient to ensure that the advantageous or synergistic effect is achieved. It will be appreciated that the preferred method and order of administration and the respective dosage amounts and regimes for each component of the combination will depend on the particular proteasome inhibitor and the HDAC inhibitor being administered, the route of administration of the combination, the disease being treated and the particular host being treated. The optimum method and order of administration and the dosage amounts and regime can be readily determined by those skilled in the art using conventional methods and in view of the information set out herein.

The present invention further relates to a product containing as first active ingredient a proteasome inhibitor, and as second active ingredient a HDAC inhibitor, as a combined preparation for simultaneous, separate or sequential use in the treatment of patients suffering from a genetic disease linked to a conformational disorder of at least one protein, said disorder causing the proteasome degradation of the protein.

Those skilled in the art could easily determine the effective amount from the results presented hereinafter. In general, it is contemplated that a therapeutically effective amount of a proteasome inhibitor and a HDAC inhibitor would be from 0.005 mg/kg to 100 mg/kg body weight, and in particular from 0.005 mg/kg to 10 mg/kg body weight. It may be appropriate to administer the required dose as two, three, four or more sub-doses at appropriate intervals throughout the day. Said sub-doses may be formulated as unit dosage forms, for example, containing 0.5 to 500 mg, and in particular 10 mg to 100 mg of active ingredient per unit dosage form.

In view of their useful pharmacological properties, the components of the combinations according to the invention, i.e. the proteasome inhibitor and the HDAC inhibitor, may be formulated into various pharmaceutical forms for administration purposes. The components may be formulated separately in individual pharmaceutical compositions or in a unitary pharmaceutical composition containing both components.

The present invention also concerns pharmaceutical compositions containing as active ingredients at least the two compounds as defined above, as well as the use of these compounds or this composition as a medicinal product or medicament. Advantageously, such compositions comprise a therapeutically effective amount of their combination, and a pharmaceutically acceptable carrier.

The present invention therefore also relates to a pharmaceutical composition comprising a proteasome inhibitor and a HDAC inhibitor together with one or more pharmaceutically acceptable carriers or excipients.

In a specific embodiment, the term “pharmaceutically acceptable” means approved by a regulatory agency of the Federal or a state government or listed in the U.S. or European Pharmacopeia or other generally recognized pharmacopeia for use in animals, and humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject.

To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets, possibly scored or effervescent tablets, and capsules represent the most advantageous oral dosage unit form, in which case solid pharmaceutical carriers are obviously employed. They can be taken with a little water before or during the main meal.

In a particular embodiment, the composition is formulated in accordance with routine procedures as a pharmaceutical composition adapted for e.g. intramuscular or intravenous administration to human beings. Typically, compositions for intravenous administration are solutions in sterile isotonic aqueous buffer. Where necessary, the composition may also include a solubilizing agent and a local anesthetic such as lidocaine to release pain at the site of the injection. The composition is preferably in a liquid form, advantageously a saline and/or glucose composition, more advantageously a phosphate buffered saline (PBS) composition or a Ringer-Lactate solution.

As already mentioned, the amount of the therapeutic agents of the invention, i.e. the compounds as disclosed above, which will be effective in the treatment of a disease can be determined by standard clinical techniques. In addition, in vivo and/or in vitro assays may optionally be employed to help predict optimal dosage ranges. The precise dose to be employed in the formulation will also depend on the route of administration, the weight and the seriousness of the disease, and should be decided according to the judgment of the practitioner and each patient's circumstances.

However and according to a particular aspect, the fact that the proteasome inhibitor is used in combination with a compound modifying the epigenome, preferably a HDAC inhibitor, allows drastically decreasing the quantity of the proteasome inhibitor to be administered. This is very advantageous since the proteasome inhibitors are known to have side-effects especially on cellular survival and therefore are not foreseen as promising drugs for the treatment of chronic diseases. For the time being, their clinical application is limited to cancers.

In the frame of the present application, it has been shown that by adding the other active compound, the quantity or concentration of the proteasome inhibitor can be reduced by a factor 2, 3, 4, 5 or even 6, 7, 8, 9 or 10. In other words, the effective quantity of the proteasome inhibitor in the presence of the other compound is 2, 3, 4, 5 or even 6, 7, 8, 9 or 10 times inferior to its effective quantity in the absence of said other compound.

According to a preferred embodiment, when combined in the same composition, the proteasome inhibitor is present in an amount inferior to its amount in a composition not comprising a HDAC inhibitor.

According to a preferred embodiment, the proteasome inhibitor can be used at a concentration inferior or equal to its half maximal inhibitory concentration (IC₅₀).

In other words, the quantity of the proteasome inhibitor is chosen so that it inhibits less than 50% of the proteasome activity, advantageously less than 40%, 30% or even 20%. The inhibition of the proteasome activity can be monitored based on the measurement of the percent of chymotrypsine-like activity as described in the examples, or based on the measurement of the trypsin-like or caspase-like activity of the proteasome.

Suitable administration should allow the delivery of a therapeutically effective amount of the therapeutic product to the target tissues, depending on the disease.

Available routes of administration are topical (local), enteral (system-wide effect, but delivered through the gastrointestinal (GI) tract), or parenteral (systemic action, but delivered by routes other than the GI tract). In some embodiments, the preferred route of administration is generally enteral which includes oral administration. According to other embodiments, it can be a parenteral administration, especially via intramuscular (i.e. into the muscle) or systemic administration (i.e. into the circulating system). In this context, the term “injection” (or “perfusion” or “infusion”) encompasses intravascular, in particular intravenous (IV), and intramuscular (IM) administration. Injections are usually performed using syringes or catheters.

According to one embodiment, the composition is administered orally, intramuscularly, intraperitoneally, subcutaneously, topically, locally, or intravascularly, advantageously orally and/or intravenously.

According to a preferred embodiment, the combination or composition according to the invention is administered daily, for example once per day. The treatment can last several weeks, several months, several years or even for the whole life.

As already stated, the patient is advantageously a human, particularly a new born, a young child, a child, an adolescent or an adult. The therapeutic tool according to the invention, however, may be adapted and useful for the treatment of other animals, particularly pigs, mice, pets such as dogs, farm animals or macaque monkeys.

As already mentioned, the present invention relates to the treatment of genetic diseases linked to a conformational anomaly of at least one protein causing the cellular degradation thereof, in particular its proteasome degradation. Such a disease can be easily identified since it means that it can be at least partially alleviated by administration of proteasome inhibitors such as bortezomib, MG132 and carfilzomib. This can be tested as disclosed in the examples in relation to SG models.

Genetic diseases are, by definition, diseases resulting from one or a plurality of mutations in one or a plurality of genes. Advantageously, the present application aims at monogenic diseases, that is, diseases linked to a single gene.

The mutations responsible for the conformational disorder of the resulting protein may be point mutations. However, the conformational disorder may be linked to mutations which are larger than points, for example, the deletion of a codon in the gene which codes a protein which is still at least partially active if it is not degraded.

In the context of the invention, “conformational disorder of at least one protein” or “protein conformational disorder” means the fact that the protein causing the disease is misfolded, due to the presence of at least one mutation in the gene encoding it, said disorder causing the degradation of said protein by the cell, especially by the proteasome pathway. The targeted pathologies can thus be easily identified, for example, by means of antibodies directed against the mutated protein. Indeed, even if it is correctly expressed (which may be verified at the transcript level), it is poorly detected by means of such an antibody, since it is at least partially degraded.

According to one embodiment, a disease to be treated according to the invention is a disease which can be treated using a proteasome inhibitor.

According to an embodiment, the disease to be treated according to the invention is not a cellular proliferation disorder/disease, especially cancer, e.g. multiple myeloma, leukemia, lymphoma, breast cancer, lung cancer or liver cancer.

According to another embodiment, the disease to be treated according to the invention is not a protein deposition disorder/disease (PDD), especially a neurodegenerative disorder, e.g. Wilson's disease, spinocerebellar ataxia, prion disease, Parkinson's disease, Huntington's disease, familial amytrophic lateral sclerosis, amyloidosis, Alzheimer's disease, Alexander's disease, alcoholic liver disease, cystic fibrosis, Pick's disease, or Lewy body dementia. A protein deposition disease can be defined as a disease wherein the protein undergoes pathogenic conformation, self-assembles (aggregates) and deposits in various tissues to provoke disruption of tissue integrity and function, and cell death. In a disease to be treated in the frame of the invention, the protein which undergoes pathogenic conformation is degraded by the proteasome.

In the context of the invention, the provided solution relies on the use of the claimed combination, to avoid the degradation of mutated proteins, to ensure their addressing in the final cellular compartment, and thus to restore a normal phenotype.

According to a specific aspect, the protein having the conformational disorder and submitted to the cellular degradation is a protein of the membrane or associated with the membrane, possibly integrated in a protein complex.

As illustrated in the present application, the present invention is useful for the treatment of muscle pathologies, advantageously those affecting the skeletal muscles but also possibly the heart muscle.

According to a specific embodiment, the present invention aims at recessive muscular pathologies.

Advantageously, the genetic muscular disease linked to a protein conformational disorder is a muscular dystrophy, more advantageously a progressive muscular dystrophy of proximal as well as of distal type.

Among progressive muscular dystrophies, the following diseases may particularly be mentioned:
sarcoglycanopathies, that is, α-sarcoglycanopathy or LGMD-R3 linked to an α-sarcoglycan defect, β-sarcoglycanopathy or LGMD-R4 linked to a β-sarcoglycan defect, γ-sarcoglycanopathy or LGMD-R5 linked to a γ-sarcoglycan defect, or δ-sarcoglycanopathy or LGMD-R6 linked to a δ-sarcoglycan defect;
dysferlinopathies (LGMD-R2 (=LGMD2B) or Miyoshi myopathy) linked to a dysferlin defect;
calpainopathies linked to a calpain 3 defect or LGMD-R1 (=LGMD2A);
anoctaminopathies (LGMD-R12 (=LGMD2L) or Miyoshi type 3 myopathy) linked to an anoctamin 5 defect;
Limb girdle myopathy with a FKRP or LGMD-R9 (=LGMD2I), linked to a defect of the FKRP (“fukutin-related protein”).

More generally, the targeted diseases are selected from the following group:
progressive muscular dystrophies:
   implying dysferlin (DYSF)
   implying γ-sarcoglycan
   implying α-sarcoglycan
   implying β-sarcoglycan
   implying δ-sarcoglycan
   implying calpain 3
   implying anoctamin 5 (Ano5)
   implying the fukutin related protein (FKRP)
   implying fukutin (FKTN)
   implying protein-O-mannosyltransferase 1 (POMT1)
   implying protein-O-mannosyltransferase 2 (POMT2)
   implying protein-O-linked mannose β 1,2-acetylglucosaminyl-transferase (POMGT1) or other enzymes involved in the glycosylation pathway of α-dystroglycan such as GMPPB, POMK, RXYLT1, POMGNT1, POMGNT2, B4GAT1, CRPPA, B3GALNT2, ISPD
   implying caveolin 3 (Cav3)
   implying UDP-N-acetyl glucosamine 2-epimerase (GNE)
congenital muscular dystrophies (CMD):
   implying α-dystroglycan (DAGI)
   implying laminin alpha-2 (LAMA2)
   implying like-glycosyltransferase (LARGE)
   implying collagen 6A1, 6A2, or 6A3
   implying selenoprotein 1 (SEPN1)
   implying integrin alpha7 (ITGA7)
   the ryanodine receptor (RYR)
other diseases affecting the skeletal or heart muscle, possibly associated with impairments of other organs:
   arrhythmogenic right ventricular cardiomyopathy implying transmembrane protein 43 (TMEM43)
   type 4 liposdystrophy muscular dystrophy implying polymerase I and the transcript release factor (PTRF)
   the chondrodystrophic myotonia or Schwartz Jampel Syndrome implying heparan sulfate (HSPG2)
   the Danon disease implying the lysosomal-associated membrane protein 2 (LAMP2)
   Fibrodysplasia ossificans progressiva implying the type I activin A receptor (ACVR1).

According to specific embodiments, the muscular dystrophy is selected from the group consisting of congenital muscular dystrophy, Duchenne muscular dystrophy (DMD), Becker muscular dystrophy (BMD, Benign pseudohypertrophic muscular dystrophy), distal muscular dystrophy (distal myopathy), Emery-Dreifuss muscular dystrophy (EDMD), facioscapulohumeral muscular dystrophy (FSHMD, FSHD or FSH), limb-girdle muscular dystrophy (LGMD), myotonic muscular dystrophy, centronuclear myopathies, oculopharyngeal muscular dystrophy and laminin-α2-deficient congenital muscular dystrophy (Muscular Dystrophy, Congenital Merosin-Deficient, 1a/MDC1A).

For a same disease, different mutations may affect the same gene and be responsible for the conformational disorder of the encoded protein. Thus, and as examples for sarcoglycanopathies, the main point mutations listed to date are listed in document WO2008/009802.

In the context of the present application, the feasibility of the invention has for example been demonstrated in relation with the R77C mutation of α-sarcoglycan and with the C283Y and E263K mutations of γ-sarcoglycan. However, the claimed combination can be used to treat any genetic disease linked to a conformational disorder of at least one protein causing the proteasome degradation thereof.

Therefore and according to another aspect, the invention relates to a method for identifying any genetic disease, which can be cured by the claimed combination comprising the steps of.
placing a cell capable of producing the misfolded protein causing the genetic disease in contact with the combination;
determining the rate of correctly folded protein.

One embodiment comprises testing cells, advantageously muscle cells, fibroblasts or iPSc derived cells, of a patient putatively affected by a genetic disease linked to a conformational disorder of at least one protein causing the proteasome degradation thereof. As a variation, a cDNA corresponding to the patient's genotype is transfected into a cell to perform the test in vitro.

If the patient is effectively affected with such a disease, the protein having a conformational disorder is expected to be reduced or not detectable, since it has been degraded. This may easily be tested as described hereabove, particularly by means of antibodies directed against said protein.

In the presence of the combination of the invention, it is expected for the rate of correctly folded protein to increase, particularly in the detection test implemented at the previous step.

If the rate of correctly folded protein increases along with the addition of the combination of the invention, it can be concluded that it is a genetic disease linked to a conformational disorder of the tested protein, causing the proteasome degradation thereof and that the patient can be effectively treated with such a combination.

According to one aspect, the combination according to the invention is associated with other treatments for the same disease, especially another compound for treating the same disease.

According to a specific embodiment, the present invention concerns a composition, advantageously a pharmaceutical composition or a medicinal product containing a combination according to the invention and potentially other active molecules (other gene therapy proteins, chemical groups, peptides or proteins, etc.) for the treatment of the same disease or a different disease, advantageously of the same disease.

In relation to muscular dystrophies, simultaneous or sequential administration of peptides or proteins able to increase the muscle mass, such as decorin (WO2010/106295) or fibromodulin (WO2013/072587) can be envisaged.

More generally, in relation to genetic disease linked to a conformational disorder of at least one protein, a further compound able to prevent the cellular degradation of the protein can be administered simultaneously or at different times. In case of simultaneous administration, the different compounds can be associated in the same composition.

In that context, other compounds such as mannosidase I inhibitors as disclosed in Bartoli, M. et al. (2008), in particular kifunensine, can be used. Alternatively, a further proteasome inhibitor or a further compound modifying epigenome as those disclosed in WO2014/013184 can be used.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The disclosures of each and every patent, patent application, and publication cited herein are hereby incorporated herein by reference in their entirety.

Without further description, it is believed that one of ordinary skill in the art can, using the preceding description and the following illustrative examples, make and utilize the compounds of the present invention and practice the claimed methods.

### Examples

The invention and its advantages are understood better from the examples shown below supporting the annexed figures. In particular the present invention is illustrated with regard to the effect of various combinations of proteasome inhibitors (bortezomib, MG132 or carfilzomib) and HDAC inhibitors (givinostat, belinostat or tubacin) in an in vitro model for LGMD2D (or LGMD-R3) linked to mutation R77C in the gene encoding α-sarcoglycan (α-SG) and for LGMD2C (or LGMD-R5) linked to mutations C283Y or E263K in the gene encoding γ-sarcoglycan (γ-SG). These examples are not however in any way limiting.

### BRIEF DESCRIPTION OF THE DRAWINGS

FIG. 1: Effect of bortezomib treatment on R77C-α-SGmCh membrane rescue.

(A) Confocal images of mCherry signal and α-SG detected by immunofluorescence in fibroblasts transduced with the lentivirus expressing R77C-α-SGmCh under non-permeabilized condition and treated with DMSO 0.1% or BTZ at 5 nM or 30 nM. Nuclei are labelled by Hoechst staining. Scale bar=50 μm.

(B-C) Cell viability (B), chymotrypsin-like activity of the proteasome and quantification of mCherry and membrane α-SG positive fibroblasts (C) following fibroblasts treatment with increasing concentrations of BTZ.

BTZ=bortezomib.

FIG. 2: Effect of givinostat.

(A) mCherry fluorescent signal and α-SG staining in non permeabilized condition in fibroblasts overexpressing R77C-α-SGmCh treated with 0.10% DMSO or 10 μM givinostat alone or in combination with 5 nM BTZ. Nuclei are labelled by Hoechst staining (blue). Scale bar=50 μm.

(B-D) Quantification of mCherry and membrane α-SG positive fibroblasts (B), cell viability (C) or chymotrypsin-like activity of the proteasome (D) following fibroblasts treatment with increasing concentrations of givinostat alone or in combination with 5 nM BTZ.

BTZ=bortezomib.

FIG. 3: Effect of belinostat.

(A) mCherry fluorescent signal and α-SG staining in non permeabilized condition in fibroblasts overexpressing R77C-α-SGmCh treated with 0.1% DMSO or 10 μM belinostat alone or in combination with 5 nM BTZ. Nuclei are labelled by Hoechst staining (blue). Scale bar=50 μm.

(B-D) Quantification of mCherry and membrane α-SG positive fibroblasts (B), cell viability (C) or chymotrypsin-like activity of the proteasome (D) following fibroblasts treatment with increasing concentrations of belinostat alone or in combination with 5 nM BTZ.

BTZ=bortezomib

FIG. 4: Combinatory effect of givinostat with bortezomib

(A-B) Quantification of chymotrypsin-like activity of the proteasome (A) and mCherry and membrane α-SG expression (B) in fibroblasts overexpressing R77C-α-SGmCh and treated with increasing concentrations of BTZ in the absence or in the presence of 10 nM, 30 nM, 100 nM, 300 nM, 1 μM, 3 μM or 10 μM givinostat. Values are expressed as percentage of the response induced by 0.10% DMSO (A) or as percentage of the maximal response induced by BTZ (B).

(C) EC₅₀ of BTZ and combination treatments with givinostat on membrane α-SGmCh membrane rescue and IC₅₀ of BTZ and combination treatments with givinostat on proteasome activity.

Bortezomib=BTZ

FIG. 5: Combinatory effect of the proteasome inhibitor CFZ or MG132 with givinostat.

Quantification of mCherry and membrane α-SG positive cells (A, B) and representatives immunofluorescence images (C) following treatment with increasing concentrations of givinostat in presence or in absence of 10 nM carfilzomib (A, C) or 100 nM MG132 (B, C).

BTZ=bortezomib, CFZ=carfilzomib,

FIG. 6: Evaluation of the effect of givinostat and bortezomib on γ-SG mutated proteins.

SGCG−/− fibroblasts were transduced with lentivirus expressing C283Y-γ-SGmCh or E263K-γ-SGmCh constructs and treated with DMSO (0.1%), BTZ (5 nM and 30 nM) or givinostat (10 μM) in presence or in absence of 5 nM BTZ for 24 hours. Membrane γ-SG expression was evaluated by immunofluorescence in non-permeabilized condition. (A) Images of mCherry fluorescent signal (red) and membrane γ-SG staining (green). Nuclei are labelled by Hoechst staining (blue). Scale bar=50 μm. (B-C) Quantification of mCherry and membrane C283Y-γ-SG (B) and E263K-γ-SG (C) positive fibroblasts.

BTZ=bortezomib.

FIG. 7: Combinatorial mechanism of action of givinostat and bortezomib.

(A) Schematic representation of proteasomal and lysosomal degradation pathways of misfolded proteins.

(B) Western Blot analysis of ubiquitinated proteins (top panel), P62 and LC3BI/II (middle panel) and acetylated α-tubulin (bottom panel) expression in fibroblasts overexpressing R77C-α-SGmCh and treated with DMSO 0.1%, BTZ (5 nM or 30 nM), or givinostat (10 μM) in presence or in absence of 5 nM BTZ for 24 hours.

BTZ=bortezomib.

FIG. 8: Combinatory effect of tubacin with bortezomib

(A) mCherry fluorescent signal and α-SG staining in non permeabilized condition in fibroblasts overexpressing R77C-α-SGmCh treated with 10 μM tubacin alone or in combination with 5 nM BTZ. Nuclei are labelled by Hoechst staining (blue). Scale bar=50 μm.

(B-C) Quantification of mCherry and membrane α-SG positive fibroblasts (B), and cell viability (C) following fibroblasts treatment with increasing concentrations of tubacin alone or in combination with 5 nM BTZ.

(D) Western Blot analysis of ubiquitinated proteins (middle panel), α-SG (top panel) and acetylated α-tubulin (bottom panel) expression in fibroblasts overexpressing R77C-α-SGmCh and treated with DMSO 0.1%, BTZ (5 nM or 30 nM), or tubacin (10 μM) in presence or in absence of 5 nM BTZ for 24 hours.

BTZ=bortezomib.

### MATERIALS AND METHODS

### Plasmid Cloning and Mutagenesis.

The α-sarcoglycan fusion protein was designed based on the human SGCA consensus coding sequence (CCDS) found in the NCBI portal (Gene ID: 6442, CCDS number 45729.1) and the mCherry sequence: The linker, in the amino-acid form of -GGGGS-, was chosen as a flexible type linker that also increased stability/folding of the fusion proteins. The fusion protein nucleotide sequence was synthesized by Genecust and cloned in a vector plasmid prepared for lentivirus production. The final construct, termed α-SGmCh, was driven by the cytomegalovirus (CMV) promoter. The R77C mutation was generated based on the 229C>T nucleotide change of SGCA gene. Mutagenesis was performed using the QuikChange XL Site-Directed Mutagenesis kit (200516, Agilent) according to manufacturer's instructions.

The primers used to introduce the R77C mutation were:

Forward
(SEQ ID NO: 1)
5′-GCCCCGGTGGCTCTGCTACACCCAGCGC-3′
and
Reverse
(SEQ ID NO: 2)
5′-GCGCTGGGTGTAGCAGAGCCACCGGGGC-3′.

The γ-sarcoglycan fusion protein was designed based on the human SGCG consensus coding sequence (CCDS) found in the NCBI portal (Gene ID: 6445, CCDS number 9299.1) and the mCherry sequence: The linker in the amino-acid form of -GGGGS was chosen as a flexible type linker that also increased stability/folding of the fusion proteins. The fusion protein nucleotide sequence was synthesized by Genecust and cloned in a vector plasmid prepared for lentivirus production. The final construct, termed γ-SGmCh, was driven by the cytomegalovirus (CMV) promoter. The C283Y and E263K mutations were generated based on the TGc>Tac and Caa>Aaa nucleotide changes of SGCG gene, respectively. Mutagenesis was performed using the QuikChange XL Site-Directed Mutagenesis kit (200516, Agilent) according to manufacturer's instructions.

The primers used to introduce the C283Y and E263K mutations were:

For C283Y:
Forward
(SEQ ID NO: 3)
5′-ggtgtgagcaccacgtAccaggagcacaacc-3′
and
Reverse
(SEQ ID NO: 4)
5′-ggttgtgctcctggTacgtggtgctcacacc-3′
For E263K:
Forward
(SEQ ID NO: 5)
5′-gctcacagagcctctacAaaatctgtgtgtgtcc-3′
and
Reverse
(SEQ ID NO: 6)
5′-ggacacacacagatttTgtagaggctctgtgagc-3′.

### Cell Culture, Transduction and Pharmacological Treatments.

The fibroblasts used in this study were isolated from a patient biopsy obtained by the Genethon's Cell Bank. Informed consents were obtained from the parents of the patient included in this study, complying with the ethical guidelines of the institutions involved and with the legislation requirements of the country of origin.

Fibroblasts were transduced with a pBABE-puro-based retroviral vector containing sequence encoding the catalytic subunit of human telomerase reverse transcriptase (hTERT) and then selected in the presence of puromycin (1 mg/ml) for 10 days, as previously described (Chaouch, S. et, Human gene therapy 20, 784-790 (2009)). Fibroblasts were cultured in Dulbecco's modified Eagle's medium+GlutaMAX (Invitrogen) supplemented with 10% fetal bovine serum (research grade, Sigma-Aldrich) and 1% Penicillin-Streptomycin (Invitrogen).

For overexpression of mutated α-SG and γ-SG, immortalized fibroblasts were transduced with a lentiviral vector expressing human R77C-α-SGmCh, C283Y-γ-SGmCh or E263K-γ-SGmCh with a multiplicity of infection (MOI) of 20 in the presence of 4 μg/ml of polybrene (Sigma-Aldrich). Cells were seeded on plates coated with 50 μg/ml of collagen I and maintained in a humidified atmosphere of 5% CO2 at 37-C. Twenty-four hours after seeding, cells were treated with the tested compounds (bortezomib, Selleckchem; givinostat, Selleckchem; belinostat, Selleckchem; tubacin, Selleckchem; MG132, Selleckchem; carfilzomib, Selleckchem) or the carrier dimethyl sulfoxide (DMSO, VWR). Cells were analyzed after 24 hours of treatment.

### Immunoblotting.

Twenty-four hours after seeding, the immortalized fibroblasts expressing R77C-α-SGmCh were incubated with the tested compounds or the carrier. Cells were collected after 24 hours of treatment. Proteins were extracted by cell lysis buffer (NP40 Buffer, Thermo Scientific) and Proteases Inhibitors (Complete PIC, Roche). Proteins were separated using a 3-8% Criterion™ XT tris-acetate protein gel and then transferred to PVDF membrane with a Trans-Blot Turbo Transfert system (Biorad) using the 7 min/25V program. Detection of proteins was performed using standard Odyssey protocol by incubation with the following antibodies:
Ubiquitinated proteins: Merck, 04-263;
acetylated α-tubulin: Sigma, T6793;
P62: Abcam, ab56416;
LC3BI/II: Novus, NB600-1384.

Western blot were revealed using Odyssey secondary antibodies donkey anti mouse (DAM) 680 diluted 1:10000, and donkey anti rabbit (DAR) 800 diluted 1:5000, 1H at RT and then scanned with the Odyssey machine.

### SG Localization Cell-Based Assay and Viability Test.

After 24 hours of drug treatment, cells were fixed in 4% paraformaldehyde (10 min, room temperature). Immunocytochemistry was performed in a phosphate-buffered saline (PBS) solution supplemented with 1% bovine serum albumin (BSA; Sigma) for blocking (1 hour, room temperature) and with a mouse anti-α-SG (NCL-L-a-SARC, Novocastra, Leica) or anti-γ-SG (NCL-L-g-SARC, Novocastra, Leica) for primary hybridization step (overnight, 4° C.). Cells were stained with a fluorophore-conjugated secondary anti-mouse antibody (Invitrogen; 1 hour, room temperature) and nuclei were visualized with Hoechst 33342 (Invitrogen). SG localization was analyzed with a CellInsight CX7 HCS Platform (Cellomics Inc). The first channel was used for nuclei identification, the second one for membrane SG staining identification and the third one for mCherry tag identification. Pictures were acquired with a 10× objective in high-resolution camera mode and were analyzed using the colocalization bioapplication. The number of cells was monitored by counting Hoechst stained cells per field allowing quantification of the cell viability.

### Proteasomal Activity Assay.

Fibroblasts were seeded in 384 well plates and treated with tested combinations as indicated for 12 hours. Proteasome-Glo™ or chymotrypsin-like cell-based assay reagents were added according to manufacturer instructions (Promega). Luminescence was read using a CLARIOstar® microplate reader (BMG Labtech).

### Statistical Analysis.

Data are presented as means±SD. Statistical analysis was performed using the Student's t test. Curve-fitting, IC₅₀ and EC₅₀ determinations were performed using GraphPad Prism (v5.0.3).

### Results

### 1/Validation of the R77C-α-SGmCh Cell Model.

In order to easily evaluate the membrane localization of the R77C-α-SG mutant in the heterologous condition, a system was generated that allowed the simultaneous identification of the positive cells for the exogenous protein and the quantification of the amount of protein in the cell membrane. A fusion protein was created, consisting of the coding sequence for human R77C-α-SGfused with an mCherry (mCh) fluorescent reporter at the C-terminus. A flexible linker was inserted between the two coding sequences to avoid protein interference in protein maturation and folding. The construct, hereafter termed R77C-α-SGmCh, was placed under the transcriptional control of the cytomegalovirus (CMV) promoter and inserted into a lentivirus backbone. Immortalized fibroblasts from a LGMD-R3 patient were used as cellular model because of their absence of endogenous α-SG.

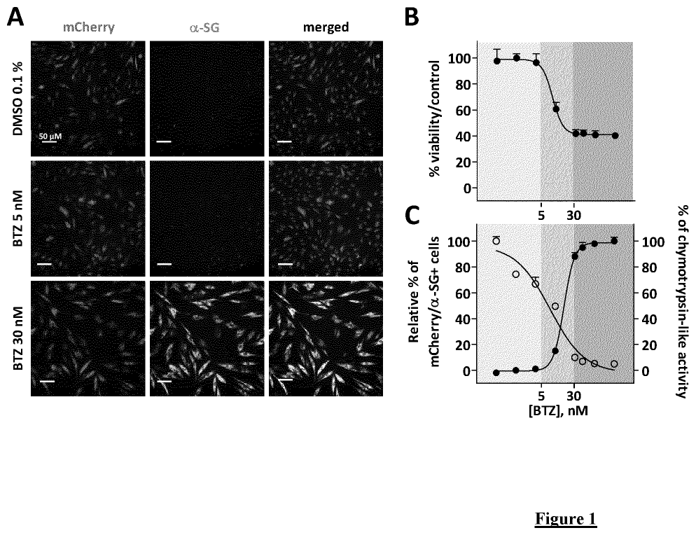

Confocal analysis of α-SG immunofluorescence (IF) revealed no detectable membrane localization of the R77C mutant protein in non-permeabilized condition (FIG. 1A, top panels), indicating retention of the protein. The relevance of the model was validated by showing the rescue of R77C-α-SG localization in the plasma membrane, following a treatment with 30 nM of the proteasome inhibitor bortezomib (BTZ) (FIG. 1A, bottom panels). Dose-response experiments quantifying the toxicological profile of BTZ (FIG. 1B) as well as the IC₅₀ on proteasome activity and the EC₅₀ on R77C-α-SG rescue (FIG. 1C) allowed to identify a subtoxic dose of BTZ (5 nM) rescuing partially R77C-α-SG degradation with a limited inhibition of the proteasome activity and toxicity. This concentration of BTZ was selected for combinatorial screening to identify drugs acting in synergy for R77C-α-SG membrane rescue.

### 2/Identification of Givinostat and Belinostat as Inhibitors of Misfolded Mutated α-SG Protein Degradation.

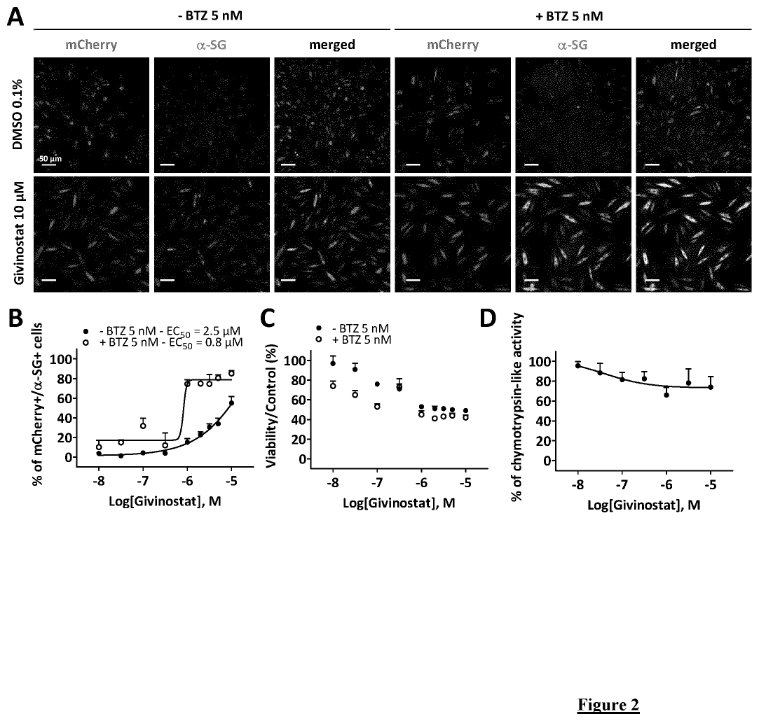

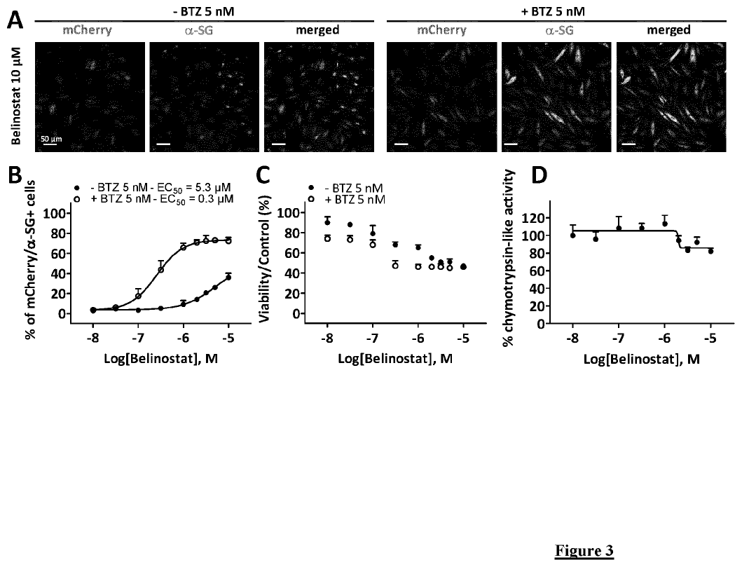

As shown in FIG. 2A and FIG. 3A, givinostat and belinostat 10 μM revealed low effect on R77C-α-SGmCh membrane rescue as detected by punctiform α-SG staining, while the addition of 5 nM BTZ induced a diffuse α-SG staining suggesting a synergy with BTZ. These observations were confirmed by dose-response experiments quantifying the presence of R77C-α-SGmCh at the membrane and revealing a significantly higher efficacy of the combined treatments (EC50 of 0.8 μM and 0.3 μM, respectively) as compared to givinostat or belinostat treatment alone (EC50 of 3.0 μM and 5.3 μM, respectively) (FIG. 2B and FIG. 3B) without additional toxicity (FIGS. 2C and 3C). Measurement of the chymotrypsin-like activity of the proteasome following increasing concentrations of givinostat or belinostat (FIGS. 2D and 3D) revealed no proteasomal inhibition suggesting a distinct mechanism of action than BTZ.

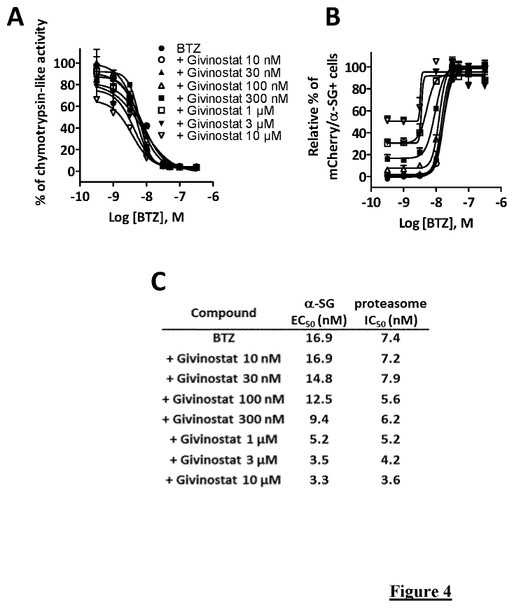

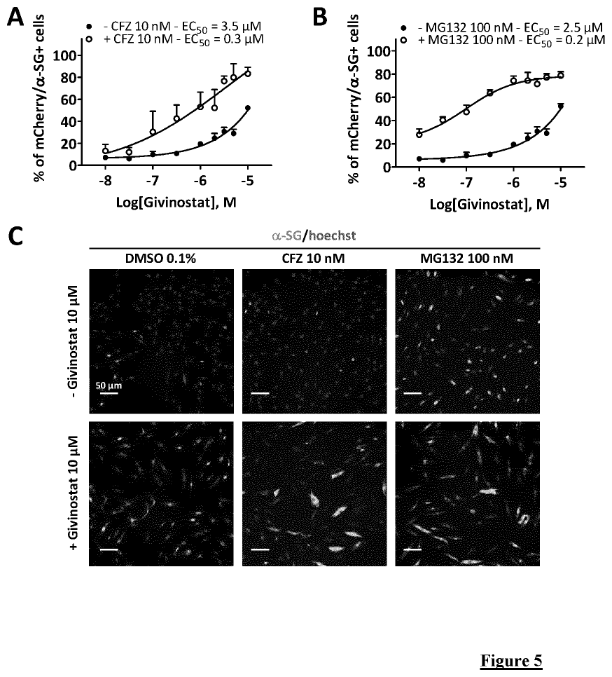

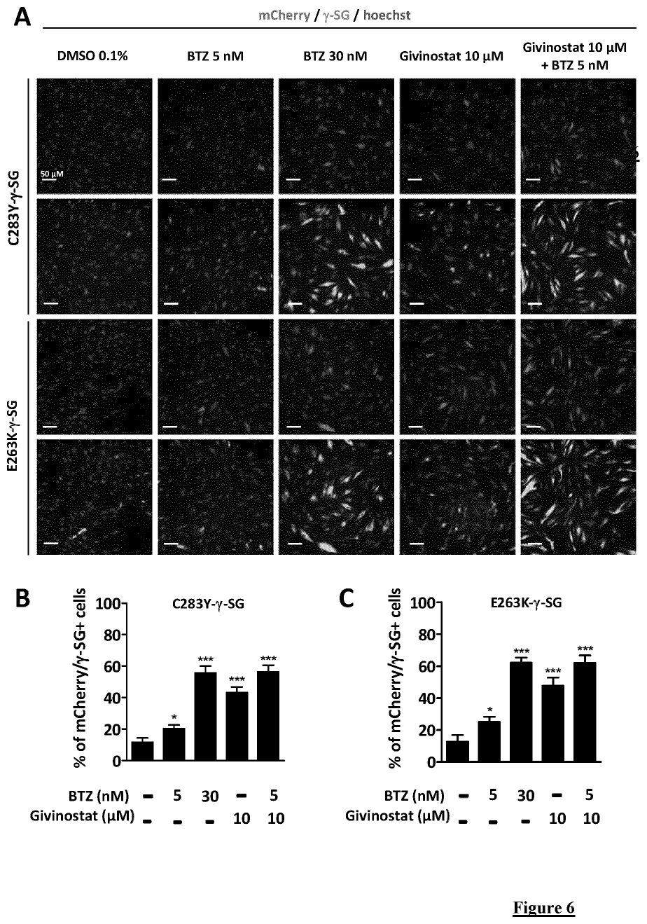

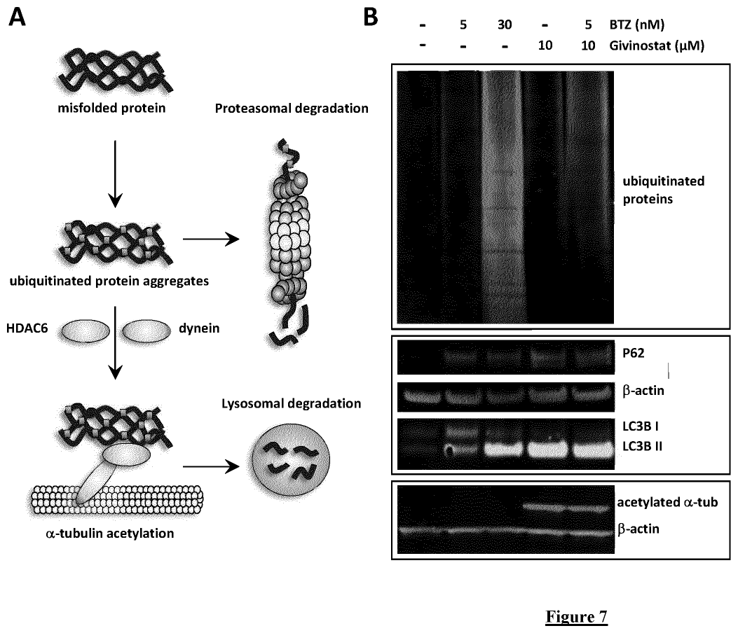

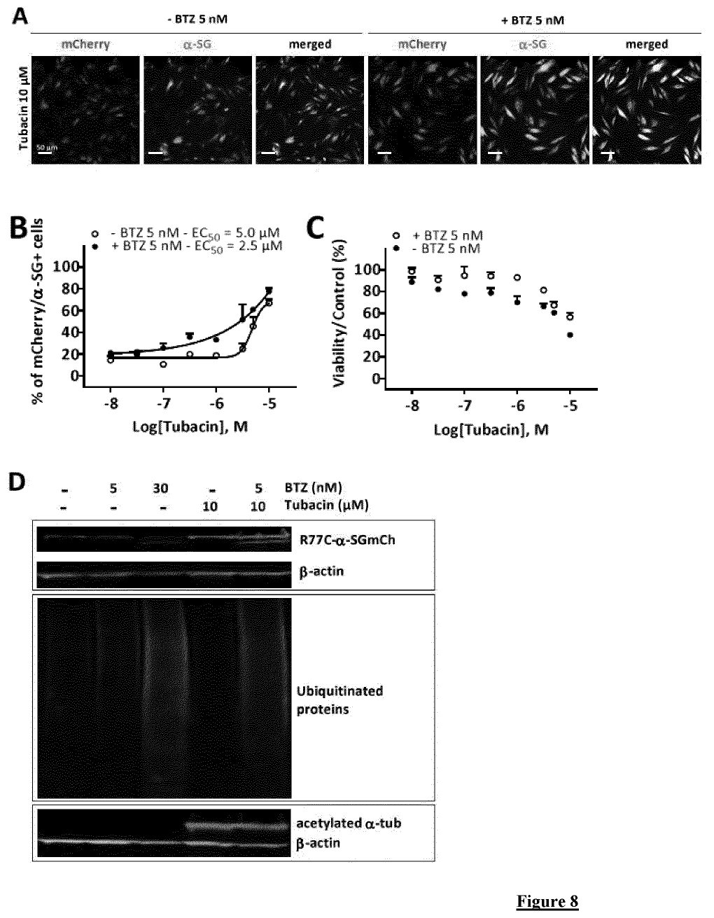

Evaluation of the effect of givinostat on the BTZ-mediated proteasome inhibition by the measurement of the chymotrypsin-like activity after treatments with increasing concentrations of BTZ in the presence of 10 nM, 30 nM, 100 nM, 300 nM, 1 μM, 3 μM or 10 μM of givinostat (FIGS. 4A and 4C) revealed no significant IC₅₀ changes induced by the different combinations, while significant EC₅₀ changes of the membrane R77C-α-SGmCh mutant protein rescue were observed (FIGS. 4B and 4C), providing evidences that givinostat had no effect on BTZ-mediated proteasome inhibition.

Similar results were obtained by the combination of givinostat with two other proteasome inhibitors respectively, carfilzomib and MG132 (FIGS. 5A, 5B and 5C). Membrane localization of R77C-α-SGmCh was observed after treatments with increasing concentrations of givinostat combined to low doses of carfilzomib (10 nM) or MG132 (100 nM) confirming their synergistic effect on R77C-α-SGmCh mutant protein rescue.

### 3/Rescue of Different Missense Mutations of SG Proteins Through Combination of Givinostat and Bortezomib Treatment.

The effect of givinostat and BTZ combination was evaluated on other different missense mutations leading to misfolded γ-SG frequently reported in LGMD-R5 patients. Lentiviruses expressing the C283Y or E263K γ-SGmCh were transduced to SGCG KO fibroblasts. Immunofluorescence images analysis in non-permeabilized condition revealed the absence of detectable γ-SG staining at the membrane level in the mutant transduced cells (FIG. 6A). Evaluation of givinostat and BTZ combination revealed that these misfolded γ-SG proteins were also rescuable from proteasomal degradation with efficacies ranging between 55% and 65% of rescued cells (FIGS. 6A, 6B, and 6C).

### 4/Combinatorial Mechanism of Action of Givinostat and Bortezomib.

Studies demonstrating that proteasome inhibitors could induce autophagy as a compensatory response are well documented (FIG. 7A). Since the data of FIG. 4A indicate that givinostat does not act through proteasome inhibition, its possible action through inhibition of HDAC6 which is involved in lysosomal degradation was investigated.

As shown in FIG. 7B, combination of givinostat and BTZ induced a synergistic accumulation of ubiquitinated proteins suggesting a synergistic blockade of ubiquitinated protein degradation (FIG. 7B, top panel). Moreover increasing conversion of LC3B-I to LC3B-II with P62 accumulation following combined givinostat and BTZ treatment were observed reflecting an inhibition of the autophagy (FIG. 7B, middle panel). Finally, givinostat treatment alone or in combination with 5 nM BTZ induces the acetylation of α-tubulin suggesting an inhibitory effect of givinostat on HDAC6 activity in LGMD-R3 cells (FIG. 7B, bottom panel). As demonstrated on FIG. 7 by monitoring proteins sensitive to HDAC6, givinostat possibly combined with BTZ allows to protect these proteins from autophagic degradation.

Based on the hypothesis of an inhibitory effect of givinostat on HDAC6 activity in LGMD-R3 cells, the effect of 10 μM tubacin, a selective HDAC6 inhibitor, was examined on R77C-α-SGmCh mutant rescue. Tubacin induced a dose-response curve for membrane R77C-α-SGmCh expression and a synergistic effect with 5 nM BTZ (FIGS. 8A and 8B) with cell toxicity in the same range than givinostat (FIG. 8C). Western blot analysis confirmed increased α-SG protein levels, synergistic accumulation of ubiquitinated proteins and acetylation of a-tubulin (FIG. 8D) following combined tubacin and BTZ treatment.

### CONCLUSION

The present study identifies HDAC inhibitors as candidate molecules able to rescue SG mutant proteins from ER-retention and early degradation, and their use in combination with proteasome inhibitors which concentration can be drastically reduced.

This combination then constitutes a new therapeutic solution, more efficient and safer, for the treatment of e.g. LGMD-R3 or LGMD-R5 patients affected by missense mutations.

1. A combination of a proteasome inhibitor and a histone deacetylase (HDAC) inhibitor for use in the treatment of a genetic disease linked to a conformational disorder of at least one protein degraded by the proteasome.

2. A combination for its use according to claim 1, wherein the proteasome inhibitor is selected in the group consisting of: bortezomib, MG132 and carfilzomib, advantageously bortezomib.

3. A combination for its use according to claim 1 or 2, wherein the HDAC inhibitor inhibits HDAC6.

4. A combination for its use according to any of claims 1 to 3, wherein the HDAC inhibitor is selected in the group consisting of: givinostat (ITF2357), belinostat (PXD101), tubacin, advantageously givinostat.

5. A combination for its use according to any of claims 1 to 4, wherein the proteasome inhibitor is in a concentration inferior or equal to its half maximal inhibitory concentration (IC₅₀).

6. A combination for its use according to any of claims 1 to 5, wherein the combination is in the form of a pharmaceutical composition comprising the proteasome inhibitor and the HDAC inhibitor.

7. A combination for its use according to claim 6, wherein the proteasome inhibitor is present in an amount inferior to its amount in a composition not comprising a HDAC inhibitor.

8. A combination for its use according to any of claims 1 to 5, wherein the combination is for simultaneous, separate or sequential use.

9. A combination for its use according to any of the preceding claims, wherein the genetic disease linked to a conformational disorder of at least one protein is a muscular dystrophy.

10. A combination for its use according to claim 9, wherein the muscular dystrophy is selected from the group consisting of: sarcoglycanopathies, dysferlinopathies, anoctaminopathies, calpainopathies and dystrophies associated with a FKRP (“Fukutin-Related Protein”) disorder, advantageously a sarcoglycanopathy, more advantageously an α-sarcoglycanopathy or an γ-sarcoglycanopathy.

11. A combination for its use according to claim 9, wherein the protein is selected from the group consisting of: sarcoglycan, advantageously α-sarcoglycan or γ-sarcoglycan, dysferlin, anoctamin 5, Fukutin-Related Protein (FKRP) and calpain 3.

12. A combination for its use according to any of the preceding claims, wherein the combination is associated with other treatments for the same disease.

13. A combination for its use according to any of the preceding claims, wherein the combination comprises a further compound for treating the same disease.

14. A combination for its use according to claim 13, wherein the further compound is another proteasome inhibitor, a mannosidase I inhibitor, or another compound modifying epigenome.

15. A combination for its use according to any of the preceding claims, wherein the combination is administered orally, intramuscularly, intraperitoneally, subcutaneously, topically, locally, or intravascularly, advantageously orally and/or intravenously.
